(19) United States
(12) Patent Application Publication
(10) Pub. No.: US 2020/0105386 A1
(43) Pub. Date: Apr. 2, 2020

(54) METHOD AND SYSTEM TO AUTOMATE THE DESIGNATION OF THE INTERNATIONAL CLASSIFICATION OF DISEASE CODES FOR A PATIENT

(71) Applicant: Modernizing Medicine, Inc., Boca Raton, FL (US)

(72) Inventors: Michael Sherling, Boca Raton, FL (US); Daniel Cane, Boynton Beach, FL (US)

(73) Assignee: Modernizing Medicine, Inc., Boca Raton, FL (US)

(21) Appl. No.: 16/507,521

(22) Filed: Jul. 10, 2019

Related U.S. Application Data
(63) Continuation of application No. 15/102,888, filed on Jun. 9, 2016, now abandoned.
(60) Provisional application No. 61/915,181, filed on Dec. 12, 2013.

(51) Int. Cl. G16H 10/60; G16H 15/00; G06N 5/02

(52) U.S. Cl. CPC G16H 10/60; G06N 5/025; G16H 15/00

(57) ABSTRACT

A system for automating the designation of a disease classification code. In one embodiment, the system includes a rules engine; an input device for entering patient information in communication with the rules engine; an output device in communication with the rules engine; and a database in communication with the rules engine, the database comprising a plurality of rules for providing a disease classification code in response the input patient information, wherein each of the plurality of rules comprises at least one alpha-numeric character corresponding to a digit in the disease classification code; and wherein at least one of the plurality of rules comprises at least one alias rule comprising at least a second alpha-numeric character corresponding to a second digit in the disease classification code.

### RELATED APPLICATIONS

This application claims priority from U.S. Provisional Application 61/915,181 filed Dec. 12, 2013 owned by the assignee of the present application. The contents of this provisional application is herein incorporated by reference its entirety.

### FIELD OF THE INVENTION

The invention relates generally to a system and method of automating the designation of the International Classification of Disease (ICD) codes for a patient, and more specifically for automating the ICD designation based on anatomical locations, disease severity and visit related metadata.

### BACKGROUND OF THE INVENTION

The International Classification of Disease is the standard diagnostic tool for designating human disease and is used to monitor the incidence and prevalence of various health related issues. Each disease or health problem or related issue is designated by an alpha-numerical code approved by the World Health Organization (WHO). Various jurisdictions use these codes for statistical analysis. For example, WHO member states use the codes to classify diseases or other health problems and to store and record the resulting patient information so as to compile national mortality and morbidity statistics. Some of the countries of the WHO use the codes for reimbursement and resource allocation.

In the United States ICD-10 is a version of the classification codes that is mandated for use by Oct. 1, 2014 for all US physicians in the billing of their office and hospital visits. ICD-10 is a much more complex classification than the previous ICD-9 classification making it especially cumbersome to use for physicians, who previously used ICD-9.

What is needed is a way to automate the designation of the proper ICD code automatically, based on patient information. The present invention addresses this need.

### SUMMARY OF THE INVENTION

In one aspect, the invention relates to a system for automating the designation of a disease classification code. In one embodiment, the system includes a rules engine; an input device for entering patient information in communication with the rules engine; an output device in communication with the rules engine; and a database in communication with the rules engine, the database comprising a plurality of rules for providing a disease classification code in response the input patient information, wherein each of the plurality of rules comprises at least one alpha-numeric character corresponding to a digit in the disease classification code; and wherein at least one of the plurality of rules comprises at least one alias rule comprising at least a second alpha-numeric character corresponding to a second digit in the disease classification code.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The structure and function of the invention can be best understood from the description herein in conjunction with the accompanying figures. The figures are not necessarily to scale, emphasis instead generally being placed upon illustrative principles. The figures are to be considered illustrative in all aspects and are not intended to limit the invention, the scope of which is defined only by the claims.

FIG. 1 is a block diagram of an embodiment of a system constructed in accordance with the invention;

FIG. 2 is a listing of the structure of a rule and its aliases;

FIG. 3 is a screenshot of a diagnosis window;

FIG. 4 is a screenshot of a popup window within the diagnosis window;

FIG. 5 is a screenshot of a multiple diagnosis window;

FIG. 6 is a screenshot of an associated diagnosis window;

FIG. 7 is a screenshot of a single diagnosis with multiple locations window;

FIG. 8 is a screenshot of the billing window accompanying the diagnosis window of FIG. 7;

FIG. 9 is an embodiment of a rule tree for a specific disease;

FIG. 10 is a screenshot of a diagnosis window for a fracture; and

FIG. 11 is an embodiment of a billing window for FIG. 10.

### DESCRIPTION OF THE PREFERRED EMBODIMENT

In the present embodiment, as described in an American Medical Association fact sheet, ICD-10 codes range from 3-7 characters in length with many combinations of alphabetic or numeric characters (digit 1 is alphabetic; digits 2 and 3 are numeric; digits 4-7 are alpha or numeric). This means there are over 68,000 available ICD-10 codes instead of 13,000 ICD-9 codes previously available. This increase makes it difficult to memorize the codes or create a document with even the subset of the codes a physician typically uses.

ICD-10 codes are more complicated because they also factor in laterality along with body location in classifying diagnoses. As a result, instead of just diagnosing someone with folliculitis, a common skin condition, the physician now needs to know which side of the body and what part of the body the folliculitis occurs.

Additionally, ICD-10, unlike ICD-9, may factor in the severity of a disease, and can have an associated diagnosis that relates to the etiology of a given condition. For example, a leg infection called cellulitis (one ICD-10 code), that is caused by Staph Aureus bacteria will have a second associated ICD-10 code.

ICD-10 also can have different codes for common injuries such as sprains, strains, and fractures depending on the stage of healing of the injury, and whether the evaluation was an initial, follow-up or complication of a prior visit. Finally ICD-10 codes do not map easily back to ICD-9 codes in a one to one fashion. In many cases, one ICD-9 code can map into many ICD-10 codes depending on how many body parts are affected, and in other cases, many ICD-9 Codes may map to one ICD-10 code.

The medical records industry's approach to the ICD-10 transition has typically been one of general equivalence mapping. In general equivalence mappings (GEMs) (also termed cross-walks) medical record vendors translate ICD-9 codes into ICD-10 codes at the point of care. So instead of creating a native ICD-10 interface, these systems permit physicians to select the ICD-9 code and then be given a shortened list of ICD-10 codes based on the ICD-9 code. This approach is time consuming for the physicians in several ways. First, the physician may have to select between three and over one hundred codes for every diagnosis the physician makes in order to determine the best ICD-10 code. Secondly, these cross walks may not suggest the most clinically relevant ICD code because these codes are based on claims-based billing from one ICD-9 code digits to many ICD-10 code digits. The clinical etiology of the diagnosis may get “lost” in the translation.

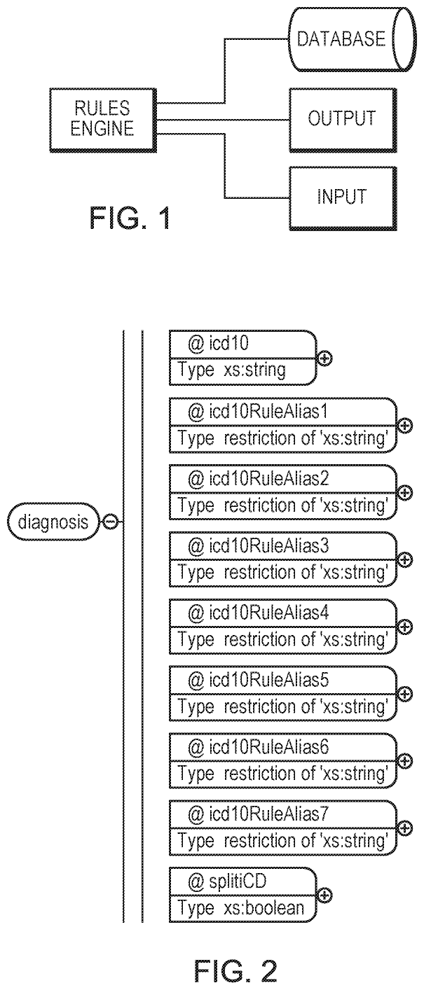

The present invention simplifies ICD-10 coding by automating the calculation of the ICD-10 through a novel clinical diagnosis decision tree using a rules engine (FIG. 1) comprising a rules schema, a processor capable of executing the rules schema, a database with general and patient specific information and an input-output device to input visit data and receive the corresponding ICD-10 code.

In the ICD-10 schema, each diagnosis contains a set of ICD-10 rules that are dynamic. These ICD-10 rules may be embedded in another rule at any specific location termed a placeholder value, within the 3-7 digit code and are denoted in the extensible markup language (XML) code as an icd10Core with icd10RuleAliases 1 through 7 as shown in FIG. 2.

In this schema, each rule may use different algorithms to determine its placeholder value but does not need to understand rules upstream (antecedent) or downstream (resultant) from its position. In one embodiment the algorithms are of three types:

In the first type, termed “Render”, the algorithm requires metadata responses from the user, for values such as: disease severity. A resulting rule, in one embodiment, appears as:

```
<mm:icd10Rule alias=“glaucomaStaging” title=“unspecified, mild,
moderate, severe, or indeterminate” renderSearch=“render” >
                  <mm:icd10Values>
                      <mm:icd10Value title=“stage unspecified”
value=“0”
      smartGuess=“true”/>
                      <mm:icd10Value title=“mild stage” value=“1”/>
                      <mm:icd10Value title=“moderate stage”
value=“2” />
                      <mm:icd10Value title=“severe stage”
value=“3”/>
                      <mm:icd10Value title=“indeterminate stage”
value=“4”/>
                  </mm:icd10Values>
            </mm:icd10Rule>
```

In the second rule type, termed “Search”, the algorithm searches all body locations from an anatomical atlas of over 40,000 anatomical zones stored in the database to return a range of numbers from 1-3 digits. A resulting rule, in one embodiment, appears as:

```
<mm:icd10Rule alias=“insuranceZoneSkinBenign” title=“Where on the
body?” renderSearch=“search” searchBy=“insuranceZone” >
                  <mm:icd10Values>
                      <mm:icd10Value title=“lip” value=“0”
fullStop=“true”/>
                      <mm:icd10Value title=“eyelid” value=“1”/>
                      <mm:icd10Value title=“ear” value=“2”/>
                      <mm:icd10Value title=“face” value=“30 ”
fullStop=“true”/>
                      <mm:icd10Value title=“nose” value=“30”
fullStop=“true”/>
                      <mm:icd10Value title=“scalp” value=“4”
fullStop=“true”/>
                      <mm:icd10Value title=“neck” value=“4”
fullStop=“true”/>
                      <mm:icd10Value title=“breast” value=“5”
fullStop=“true”/>
                      <mm:icd10Value title=“trunk” value=“5”
fullStop=“true”/>
                      <mm:icd10Value title=“hand” value=“6”/>
                      <mm:icd10Value title=“arm” value=“6”/>
                      <mm:icd10Value title=“leg” value=“7”/>
                      <mm:icd10Value title=“foot” value=“7”/>
                      <mm:icd10Value title=“upper extremity”
value=“6”/>
                      <mm:icd10Value title=“lower extremity”
value=“7”/>
                      <mm:icd10Value title=“anus” value=“5”
fullStop=“true”/>
                      <mm:icd10Value title=“genitalia” value=“5”
fullStop=“true”/>
                      <mm:icd10Value title=“scrotum” value=“5”
fullStop=“true”/>
                      <mm:icd10Value title=“penis” value=“5”
fullStop=“true”/>
                      <mm:icd10Value title=“vulva” value=“5”
fullStop=“true”/>
                      <mm:icd10Value title=“vagina” value=“5”
fullStop=“true”/>
                      <mm:icd10Value title=“other” value=“9”
fullStop=“true”/>
                      <mm:icd10Value title=“unspecified” value=“9”
smartGuess=“true” fullStop=“true”/>
                  </mm:icd10Values>
              </mm:icd10Rule>
```

The third rule type is termed “Automated” and the resulting rules in one embodiment are automated based on whether the visit type is initial or follow-up. An example of an embodiment of such a rule is:

```
<mm:icd10Rule alias=“ADS” title=“Encounter: Initial, Subsequent or
Sequela”renderSearch=“render” >
                  <mm:icd10Values>
                      <mm:icd10Value title=“Initial” value=“A”
smartGuess=“true”/>
                      <mm:icd10Value title=“Subsequent” value=“D”
/>
                      <mm:icd10Value title=“Sequela” value=“S”/>
                  </mm:icd10Values>
              </mm:icd10Rule>
```

Within each rule, a set of intelligence flags may be applied. These flags insure that only proper ICD-10 codes are calculated from the simplest scenario, where a doctor does not provide any body location or ICD-10 render metadata, to the most complex, where the doctor provides render metadata and multiple body locations. In one embodiment the intelligence flags are smartGuess and fullstop.

In one embodiment, the system always defaults to the least specified option of any rule in case the physician does not specify further detailed information. The system does this by setting the flag smartGuess=“true”. This technique insures that an accurate code is calculated at every level of the rules even assuming the worst-case scenario where the physician provides no clinical information. However, once a more specified code at any rule level is provided, as determined by body location or metadata, the unspecified flag becomes specified at that level of the rule. Additionally, rule options may truncate the down-steam rules if the rules themselves have no further specificity. These truncated rules are denoted by the flag full-stop.

For rules that search for body locations, the rules themselves can search at varying degrees of anatomic specificity, based on zones of the body (trunk, arm and leg), simple areas of the body (left leg, leg), or detailed areas of the body (left proximal medial thigh). This allows for flexibility in searching for varied ICD-10 codes across different layers of anatomy. An embodiment of such a rule is:

```
<xs:attribute name=“searchBy”>
  <xs:simpleType>
    <xs:restriction base=“xs:string”>
      <xs:enumeration value=“examDetail”/>
      <xs:enumeration value=“examSimple”/>
      <xs:enumeration value=“insuranceZone”/>
      <xs:enumeration value=“simpleThenZone”/>
    </xs:restriction>
  </xs:simpleType>
</xs:attribute>
```

For anatomical locations that are symmetrical (i.e.: upper and lower extremities, breasts, ears, eyes, eyelids), the rules can search for left or right portions of the zones, and determine whether the code should be left, right or even a single bilateral code.

If multiple body locations are selected, the clinical diagnosis itself can determine if the ICD-10 code should be just one multiple or bilateral ICD-10 code, or split into an unlimited number of ICD-10 codes based on anatomical locations. In such a case this function is designated with the intelligence flag splitICD10=“true.”

By creating a workflow in which the physician selects the diagnosis first, selects exam findings and then places them on a physical body, the system can dynamically create precise ICD-10 code without the physician having to use ICD-9 crosswalks or narrow down the list of ICD-10 options. This saves the physician time, and reduces documentation burden for billing.

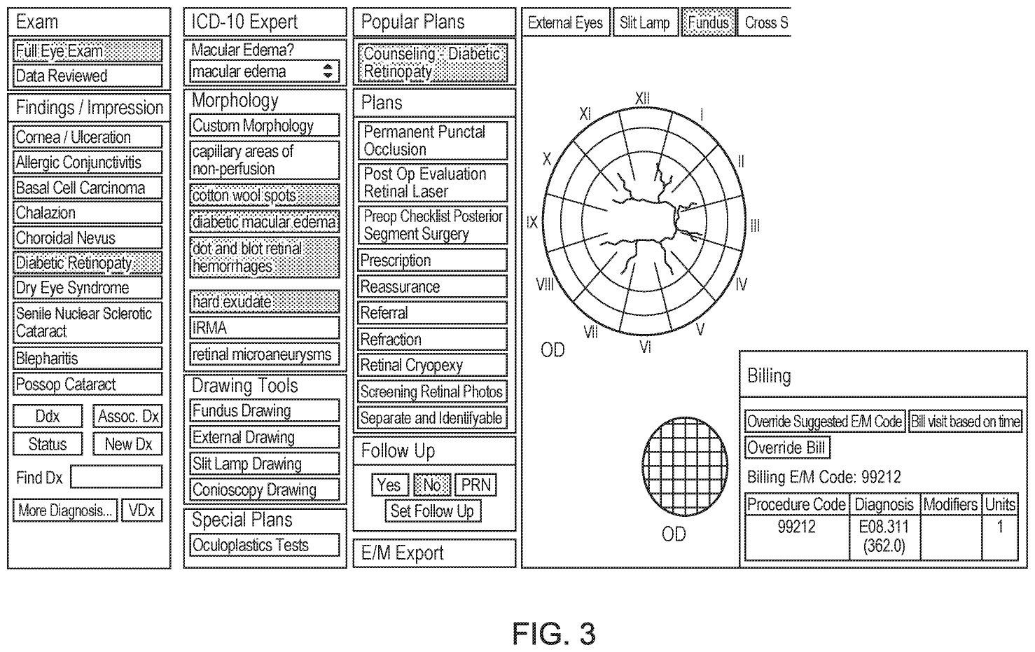

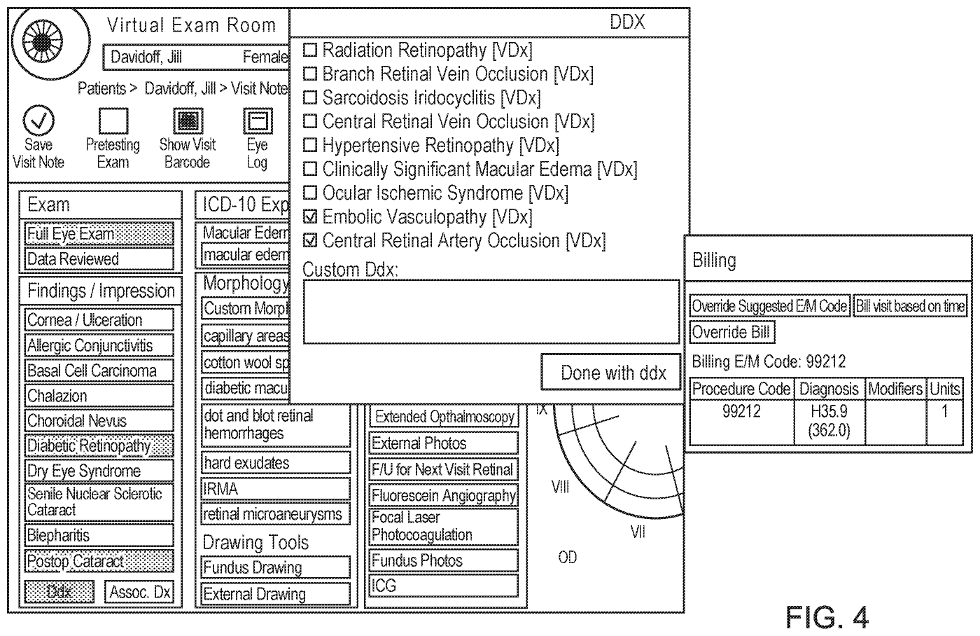

In many areas of medicine, the primary diagnosis can be uncertain. In this case, the ICD-10 rule based metadata collected for the primary diagnosis can be applied to the uncertain diagnosis; thereby reducing re-entry of metadata. In the example shown in FIG. 3, the “Diabetic Retinopathy” ICD-10 code with the Render based rule “Macular Edema” becomes E08.311. However, as soon as the Differential Diagnosis Button is pressed using the pop-up window as shown in FIG. 4, the metadata is reused to calculate Unspecified Retinal Disorder, H35.9.

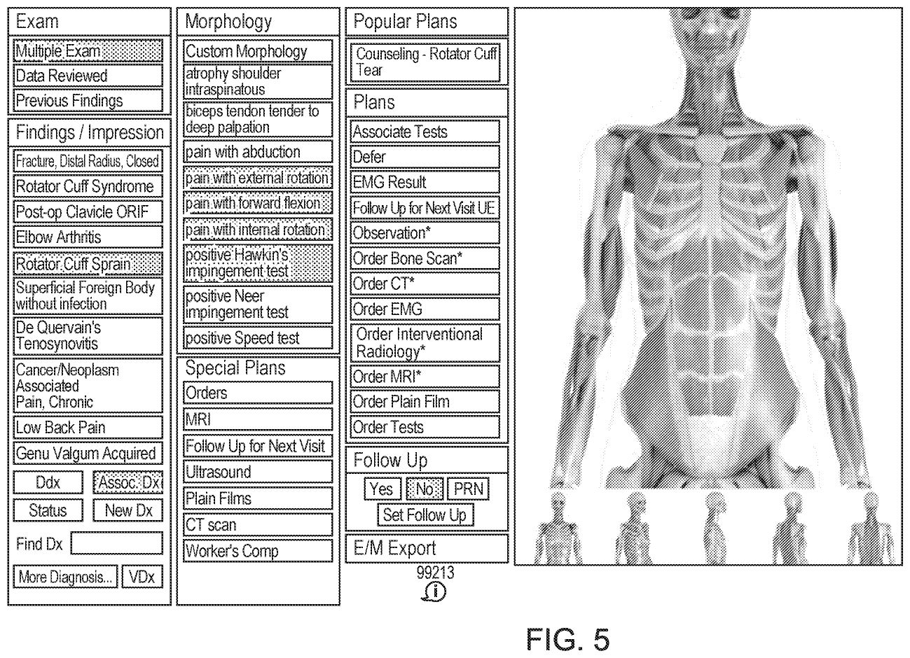

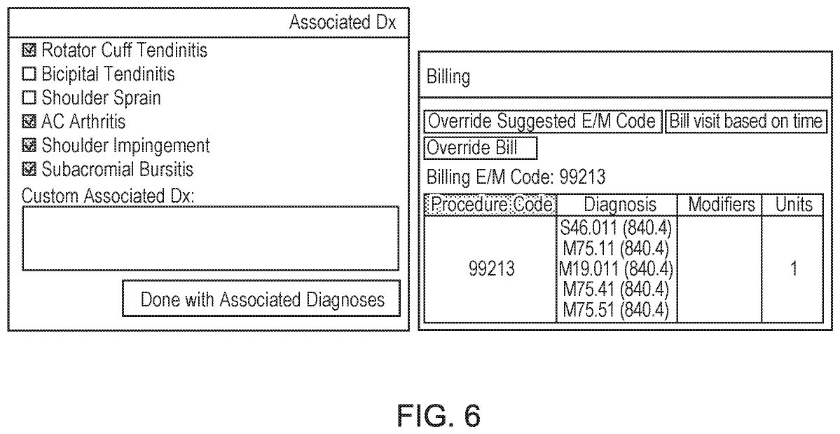

Primary diagnoses can have multiple associated diagnoses. An example, referring to FIG. 5, would be a rotator cuff sprain that has shoulder pain, AC Arthritis, Shoulder Impingement, and Subacromial Bursitis. In this case an associated diagnosis can adopt the primary diagnosis location and its related ICD-10 metadata to return the most specified code without multiple entries, FIG. 6.

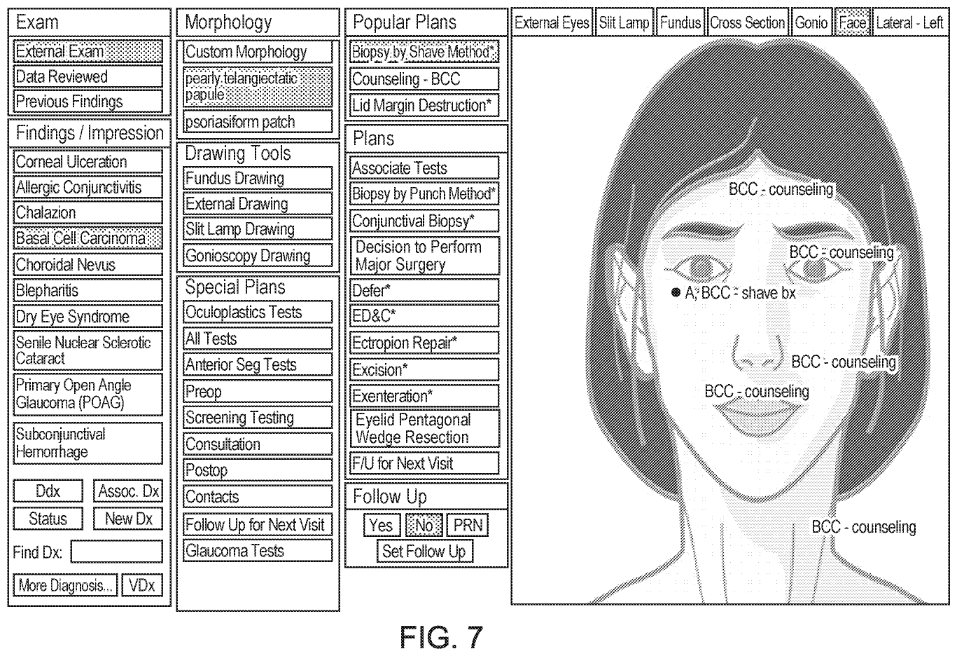

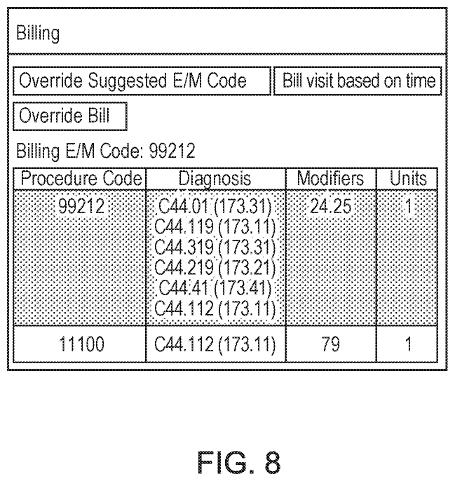

Procedures done for one diagnosis should only point to ICD-10 codes that pertain to that procedure. For example, if the physician documents multiple basal cell skin cancers in multiple areas, but only performs a biopsy on one of them, then, the system creates the biopsy current procedural terminology (CPT) code and points it to the one affected ICD-10 code out of many, FIGS. 7 and 8.

As an example of the system in use in a specific diagnosis, consider the example of the clinical diagnosis Folliculitis again. Folliculitis contains the icd10CoreRule, icd10RuleAlias4, and icd10RuleAlias6. Rules 4 and 6 are calculated based on body location metadata at different levels. As a result, the rule appears in this embodiment as:

<mm:diagnosis diagType=“infection” title=“Folliculitis” icd9=“704.8” icd10=“L02” icd10RuleAlias4=“folliculitisZone” icd10RuleAlias6=“folliculitisSimple” splitICD=“true” snoMED=“13600006”

Considering this in more detail:

The icd10CoreRule is shown as L02. This is because all folliculitis diagnoses start with L02, this code is static and is the base code for the ICD-10 dynamic set for folliculitis. The icd10RuleAlias4 designates anatomical zones. For the folliculitis zone rule, the rule first searches for any anatomic areas that are designated as “simple”. If it finds a simple area, such as buttock, it will return the value and add it to the core rule L02. If it does not, then it will search at less granular level and look for an anatomic zone such as face, scalp neck, trunk, etc. Should the folliculitis occur on the lip, breast or buttock the diagnosis will end at L02.02, L02.223, or L02.32 because of the flag fullstop=“true”. If the user does not provide a body location, the system returns the unspecified value, and returns an ICD-10 code of L02.92. If the folliculitis occurs on the trunk, upper or lower extremity, the system adds that two-digit code to L02, and goes to the next rule. The set of values thus becomes:

<mm:icd10Rule alias=“folliculitisZone” title=“Where on the body?”
renderSearch=“search” searchBy=“simpleThenZone”>

<mm:icd10Values>

| | |
|---|---|
| | <mm:icd10Value title=“lip” value=“02” |
| fullStop=“true”/> | |
| | <mm:icd10Value title=“eyelid” value=“02” |
| fullStop=“true”/> | |
| | <mm:icd10Value title=“ear” value=“02” |
| fullStop=“true”/> | |
| | <mm:icd10Value title=“face” value=“02” |
| fullStop=“true”/> | |
| | <mm:icd10Value title=“nose” value=“02” |
| fullStop=“true”/> | |
| | <mm:icd10Value title=“scalp” value=“821” |
| fullStop=“true”/> | |
| | <mm:icd10Value title=“neck” value=“12” |
| fullStop=“true”/> | |
| | <mm:icd10Value title=“breast” value=“223” |
| fullStop=“true”/> | |
| | <mm:icd10Value title=“trunk” value=“22”/> |
| | <mm:icd10Value title=“buttock” value=“32” |
| fullStop=“true”/> | |
| | <mm:icd10Value title=“hand” value=“52”/> |
| | <mm:icd10Value title=“arm” value=“42”/> |
| | <mm:icd10Value title=“axillae” value=“42”/> |
| | <mm:icd10Value title=“leg” value=“42”/> |
| | <mm:icd10Value title=“foot” value=“62”/> |
| | <mm:icd10Value title=“feet” value=“62”/> |
| | <mm:icd10Value title=“upper extremity” |
| | value=“42”/> |
| | <mm:icd10Value title=“lower extremity” |
| | value=“42”/> |
| | <mm:icd10Value title=“anus” value=“828” |
| fullStop=“true”/> | |
| | <mm:icd10Value title=“genitalia” value=“224” |
| fullStop=“true”/> | |
| | <mm:icd10Value title=“scrotum” value=“224” |
| fullStop=“true”/> | |
| | <mm:icd10Value title=“penis” value=“224” |
| fullStop=“true”/> | |
| | <mm:icd10Value title=“vulva” value=“224” |
| fullStop=“true”/> | |
| | <mm:icd10Value title=“vagina” value=“224” |
| fullStop=“true”/> | |
| | <mm:icd10Value title=“other” value=“828” |
| fullStop=“true”/> | |
| | <mm:icd10Value title=“unspecified” value=“92” |

smartGuess=“true” fullStop=“true”/>

</mm:icd10Values>

</mm:icd10Rule>

Since the only codes that have downstream rules have two digits, the next rule will be positioned at the 6 position, hence, icd10Rule6. icd10Rule6 also looks at anatomical areas this time at a more granular level called “simple”.

<mm:icd10Rule alias=“folliculitisSimple” title=“Where on the body?”
renderSearch=“search” searchBy=“examSimple”>

<mm:icd10Values>

| | |
|---|---|
| | <mm:icd10Value title=“right hand” value=“1” /> |
| | <mm:icd10Value title=“right thumb” value=“1” /> |
| | <mm:icd10Value title=“right index finger” value=“1” /> |
| | <mm:icd10Value title=“right middle finger” value=“1” |
| /> | |
| | <mm:icd10Value title=“right ring finger” value=“1” /> |
| | <mm:icd10Value title=“right small finger” value=“1” /> |
| | <mm:icd10Value title=“left hand” value=“2” /> |
| | <mm:icd10Value title=“left thumb” value=“2” /> |
| | <mm:icd10Value title=“left index finger” value=“2” /> |
| | <mm:icd10Value title=“left middle finger” value=“2” /> |
| | <mm:icd10Value title=“left ring finger” value=“2” /> |
| | <mm:icd10Value title=“left small finger” value=“2” /> |
| | <mm:icd10Value title=“right axilla” value=“1”/> |
| | <mm:icd10Value title=“left axilla” value=“2”/> |
| | <mm:icd10Value title=“right upper arm” value=“3”/> |
| | <mm:icd10Value title=“right shoulder” value=“3”/> |
| | <mm:icd10Value title=“right elbow” value=“3”/> |
| | <mm:icd10Value title=“right forearm” value=“3”/> |
| | <mm:icd10Value title=“right wrist” value=“3”/> |
| | <mm:icd10Value title=“right posterior upper arm” |
| value=“3”/> | |
| | <mm:icd10Value title=“left upper arm” value=“4”/> |
| | <mm:icd10Value title=“left shoulder” value=“4”/> |
| | <mm:icd10Value title=“left elbow” value=“4”/> |
| | <mm:icd10Value title=“left forearm” value=“4”/> |
| | <mm:icd10Value title=“left wrist” value=“4”/> |
| | <mm:icd10Value title=“left posterior upper arm” |
| value=“4”/> | |
| | <mm:icd10Value title=“right achilles skin” value=“5”/> |
| | <mm:icd10Value title=“right calf” value=“5”/> |
| | <mm:icd10Value title=“right popliteal skin” |
| value=“5”/> | |
| | <mm:icd10Value title=“right posterior thigh” |
| value=“5”/> | |
| | <mm:icd10Value title=“right thigh” value=“5”/> |
| | <mm:icd10Value title=“right shin” value=“5”/> |
| | <mm:icd10Value title=“left achilles skin” value=“6”/> |
| | <mm:icd10Value title=“left calf” value=“6”/> |
| | <mm:icd10Value title=“left popliteal skin” value=“6”/> |
| | <mm:icd10Value title=“left posterior thigh” |
| value=“6”/> | |
| | <mm:icd10Value title=“left thigh” value=“6”/> |
| | <mm:icd10Value title=“left shin” value=“6”/> |
| | <mm:icd10Value title=“right foot” value=“1”/> |
| | <mm:icd10Value title=“right great toe” value=“1”/> |
| | <mm:icd10Value title=“right 1st toe” value=“1”/> |
| | <mm:icd10Value title=“right 2nd toe” value=“1”/> |
| | <mm:icd10Value title=“right 3rd toe” value=“1”/> |
| | <mm:icd10Value title=“right 4th toe” value=“1”/> |
| | <mm:icd10Value title=“right 5th toe” value=“1”/> |
| | <mm:icd10Value title=“right heel” value=“1”/> |
| | <mm:icd10Value title=“right plantar” value=“1”/> |
| | <mm:icd10Value title=“left foot” value=“2”/> |
| | <mm:icd10Value title=“left great toe” value=“2”/> |
| | <mm:icd10Value title=“left 1st toe” value=“2”/> |
| | <mm:icd10Value title=“left 2nd toe” value=“2”/> |
| | <mm:icd10Value title=“left 3rd toe” value=“2”/> |
| | <mm:icd10Value title=“left 4th toe” value=“2”/> |
| | <mm:icd10Value title=“left 5th toe” value=“2”/> |
| | <mm:icd10Value title=“left heel” value=“2”/> |
| | <mm:icd10Value title=“left plantar” value=“2”/> |
| | <mm:icd10Value title=“back” value=“2”/> |
| | <mm:icd10Value title=“chest” value=“3”/> |
| | <mm:icd10Value title=“abdomen” value=“1”/> |
| | <mm:icd10Value title=“unspecified” value=“9” |

smartGuess=“true” fullStop=“true”/>

</mm:icd10Values>

</mm:icd10Rule>

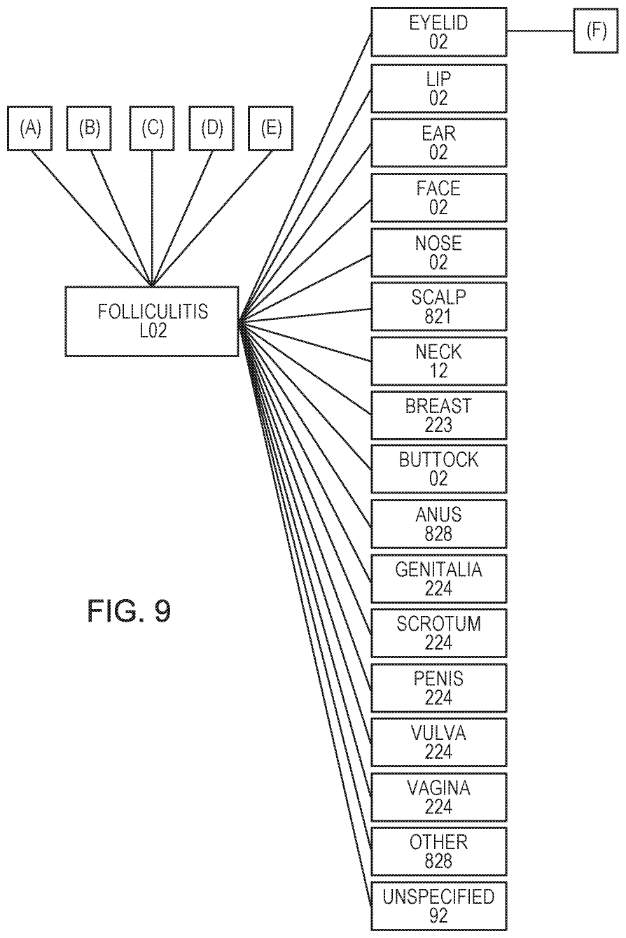

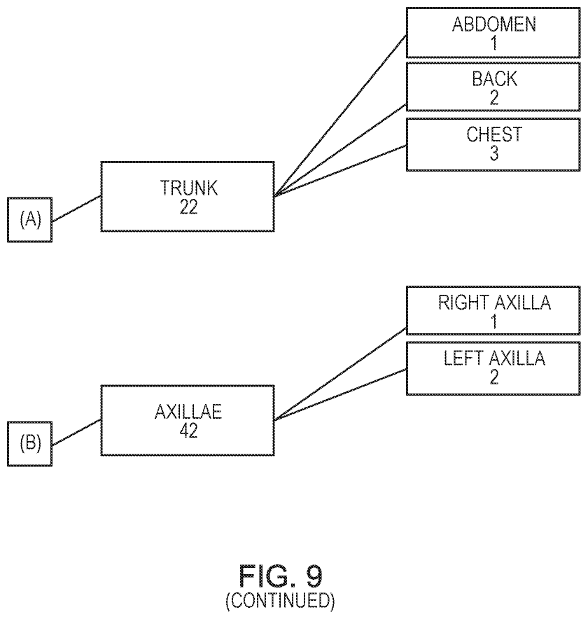

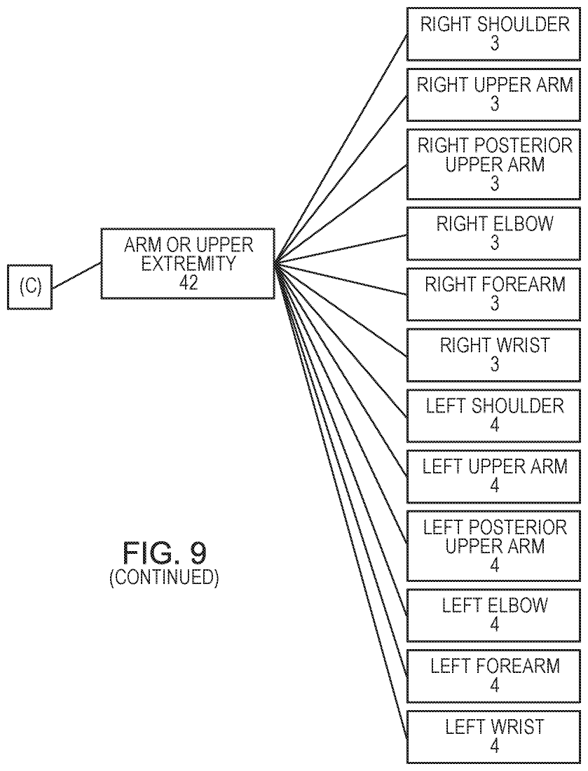

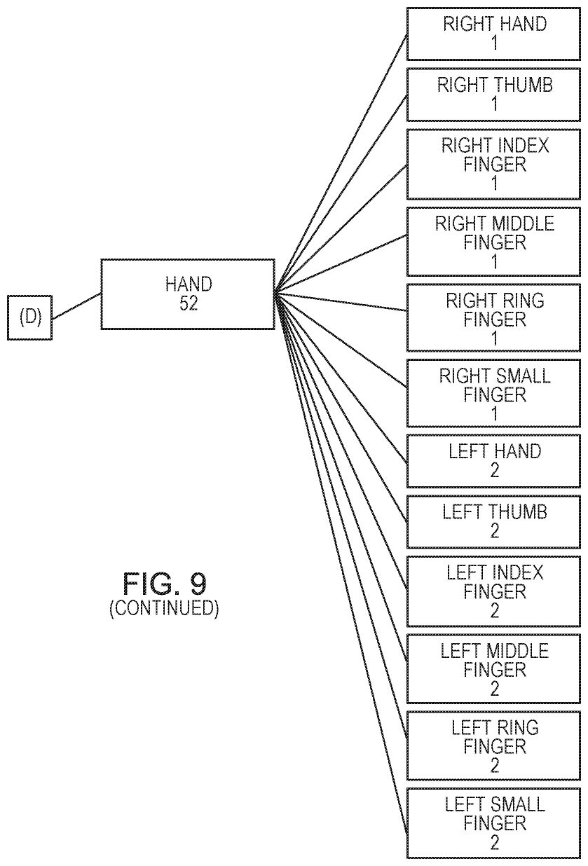

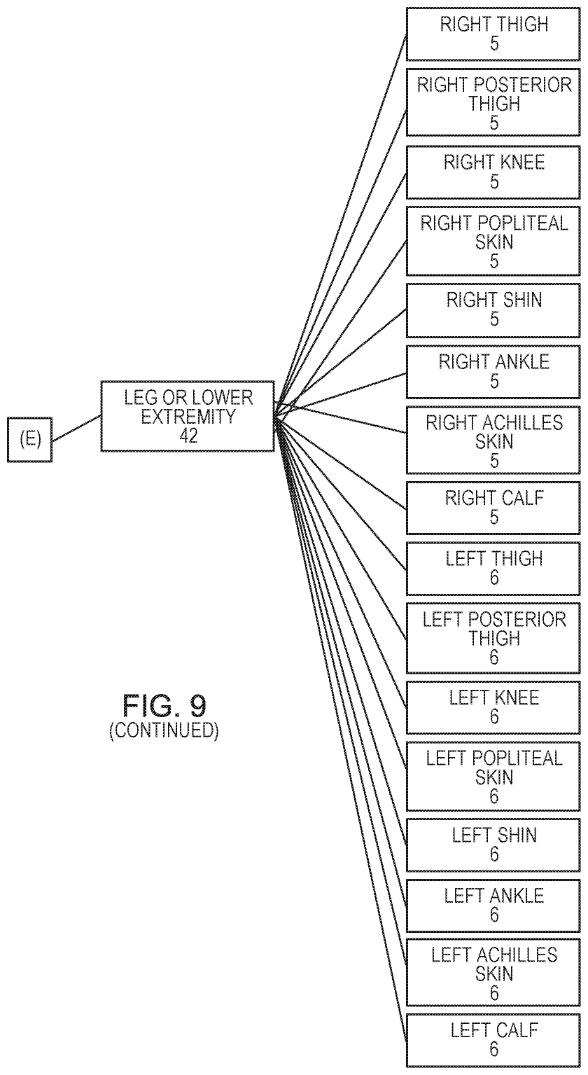

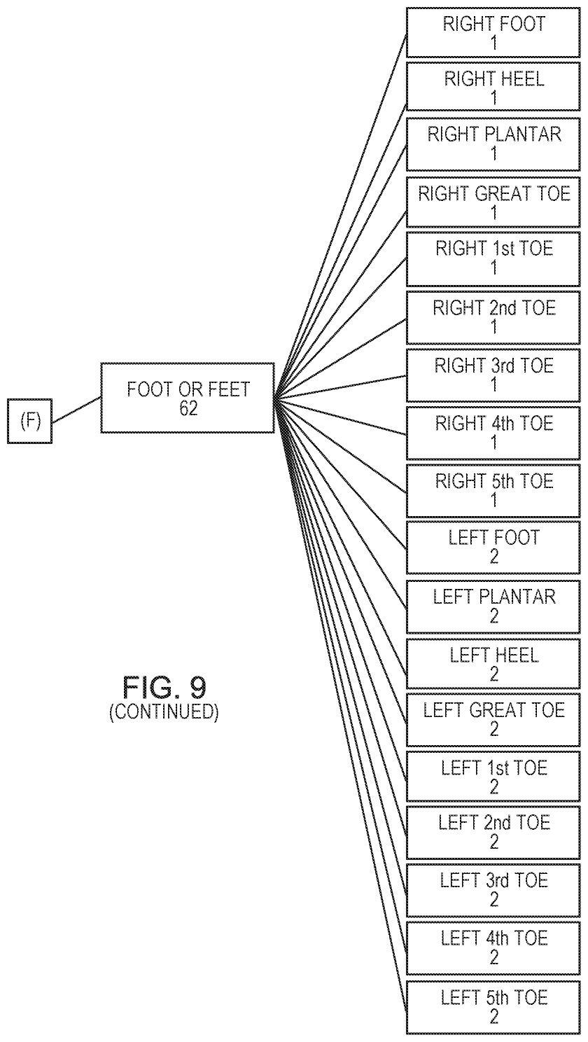

Note that if no further information is provided by the physician, the unspecified code 9 is returned to L02+two digits+9. For example, folliculitis on the trunk would be L02.229. If however, a more granular level exists, the simple level is returned. For example, the code for folliculitis on the chest is L02.223. The complete rule tree for folliculitis is shown in FIG. 9.

Since the system calculates the entire ICD-10 code set without the doctor having to search for it, the system creates accurate codes for billing and saves physician time. This is easily shown by comparing the instant system to the cross/walk GEM method. If a physician were to attempt to determine the ICD10 code for Folliculitis from an ICD9 code 704.8, the user would have to decide which among the following 3 codes to use:
L66.3—Perifolliculitis capitis abscedens
L73.1—Pseudofolliculitis barbae
L73.8—Other specified follicular disorders

This seems simple at first glance, except that NONE of the above codes would be correct for the clinical diagnosis of Folliculitis. In this case, something clinical was “lost” in translation.

In fact, the real ICD-10 code possibilities would be among the 24 below and would need to include each distinct anatomical code that applied:

L02.02—Folliculitis of face
L02.12—Folliculitis of neck
L02.221—Folliculitis/Furuncle of abdominal wall
L02.222—Folliculitis/Furuncle of back [any part, except buttock]
L02.223—Folliculitis/Furuncle of chest wall
L02.224—Folliculitis/Furuncle of groin
L02.225—Folliculitis/Furuncle of perineum
L02.226—Folliculitis/Furuncle of umbilicus
L02.229—Folliculitis/Furuncle of trunk, unspecified
L02.32—Folliculitis of buttock
L02.421—Folliculitis/Furuncle of right axilla
L02.422—Folliculitis/Furuncle of left axilla
L02.423—Folliculitis/Furuncle of right upper limb
L02.424—Folliculitis/Furuncle of left upper limb
L02.425—Folliculitis/Furuncle of right lower limb
L02.426—Folliculitis/Furuncle of left lower limb
L02.429—Folliculitis/Furuncle of limb, unspecified
L02.521—Folliculitis/Furuncle right hand
L02.522—Folliculitis/Furuncle left hand
L02.529—Folliculitis/Furuncle unspecified hand
L02.621—Folliculitis/Furuncle right foot
L02.622—Folliculitis/Furuncle left foot
L02.629—Folliculitis/Furuncle unspecified foot
L02.82—Folliculitis of other sites

Considering another diagnosis, the clinical diagnosis Closed Distal Radius Fracture contains a code icd10corerule, an icd10RuleAlias6 and an icd10RuleAlias7. A rule in this embodiment is:

<mm:diagnosis diagType=“Wrist” title=“Fracture, Distal Radius,
Closed” icd10=“S52.50” icd10RuleAlias6=“lateralityNine” splitICD=
“true” icd10RuleAlias7=“ADGKPS” icd9=“813.42”

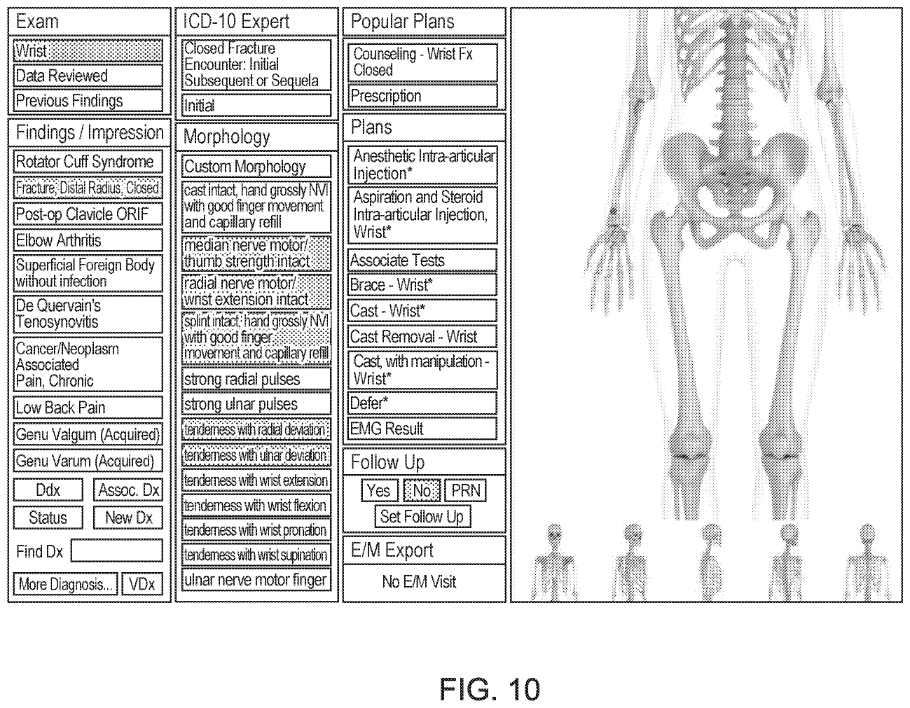

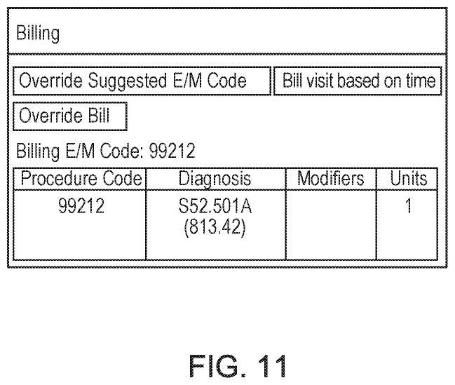

Referring to FIG. 10, because all closed distal radius fracture start with S52.50, the first dynamic rule begins at the 6^{th} position, with the alias “lateralityNine”.

<mm:icd10Rule alias=“lateralityNine” title=“Right, Left or Unspecified”
renderSearch=“search” searchBy=“examDetail” >

<mm:icd10Values>

| | |
|---|---|
| | <mm:icd10Value title=“right” value=“1”/> |
| | <mm:icd10Value title=“left” value=“2” /> |
| | <mm:icd10Value title=“unspecified” value=“9” |
| smartGuess=“true”/> | |

</mm:icd10Values>

</mm:icd10Rule>

LateralityNine searches all body locations for the presence of a left or a right. If none exist, it returns the unspecified value of 9. For a right closed distal radius fracture, the system adds the value of 1 to the core code of S52.50, and goes to rule #7.

<mm:icd10Rule alias=“ADGKPS” title=“Closed Fracture Encounter:
Initial, Subsequent or Sequela” renderSearch=“render” >

<mm:icd10Values>

| | <mm:icd10Value title=“Initial” value=“A” |
|---|---|
| smartGuess=“true”/> | |
| | <mm:icd10Value title=“Subsequent with |

Routine Healing” value=“D” />

<mm:icd10Value title=“Subsequent with

Delayed Healing” value=“G” />

<mm:icd10Value title=“Subsequent with

Nonunion” value=“K” />

<mm:icd10Value title=“Subsequent with

Malunion” value=“P” />

<mm:icd10Value title=“Sequela” value=“S”/>

</mm:icd10Values>

</mm:icd10Rule>

Rule number 7 is a render rule of ADGKPS with some built in intelligence. This rule asks the physician for more information regarding the status of the fracture. If none is given, it looks to see if the patient has ever had a clinical diagnosis of a closed distal radius fracture. If he or she has not, the system returns the value A. If he or she has, the system returns the value D. For a distal radius fracture that has routine healing and has been seen for the first time, the system calculates S52.501A, with virtually no input from the user for the new and follow-up routine healing scenarios.

If a physician were to determine the ICD-10 code for distal radius fracture using an ICD-9 to ICD-10 cross walk, instead of starting with the clinical diagnosis and using the present invention's rules, the user would have to decide among the following 195 codes:

S52501A, S52502A, S52509A, S52511A, S52512A, S52513A, S52514A, S52515A, S52516A, S52551A, S52552A, S52559A, S52561A, S52562A, S52569A, S52571A, S52572A, S52579A, S52591A, S52592A, S52599A, S59201A, S59202A, S59209A, S59211A, S59212A, S59219A, S59221A, S59222A, S59229A, S59231A, S59232A, S59239A, S59241A, S59242A, S59249A, S59291A, S59292A, S59299A
S52501D, S52502D, S52509D, S52511D, S52512D, S52513D, S52514D, S52515D, S52516D, S52551D, S52552D, S52559D, S52561D, S52562D, S52569D, S52571D, S52572D, S52579D, S52591D, S52592D, S52599D, S59201D, S59202D, S59209D, S59211D, S59212D, S59219D, S59221D, S59222D, S59229D, S59231D, S59232D, S59239D, S59241D, S59242D, S59249D, S59291D, S59292D, S59299D
S52501G, S52502G, S52509G, S52511G, S52512G, S52513G, S52514G, S52515G, S52516G, S52551G, S52552G, S52559G, S52561G, S52562G, S52569G, S52571G, S52572G, S52579G, S52591G, S52592G, S52599G, S59201G, S59202G, S59209G, S59211G, S59212G, S59219G, S59221G, S59222G, S59229G, S59231G, S59232G, S59239G, S59241G, S59242G, S59249G, S59291G, S59292G, S59299G
S52501K, S52502K, S52509K, S52511K, S52512K, S52513K, S52514K, S52515K, S52516K, S52551K, S52552K, S52559K, S52561K, S52562K, S52569K, S52571K, S52572K, S52579K, S52591K, S52592K, S52599K, S59201K, S59202K, S59209K, S59211K, S59212K, S59219K, S59221K, S59222K, S59229K, S59231K, S59232K, S59239K, S59241K, S59242K, S59249K, S59291K, S59292K, S59299K
S52501S, S52502S, S52509S, S52511S, S52512S, S52513S, S52514S, S52515S, S52516S, S52551S, S52552S, S52559S, S52561S, S52562S, S52569S, S52571S, S52572S, S52579S, S52591S, S52592S, S52599S, S59201S, S59202S, S59209S, S59211S, S59212S, S59219S, S59221S, S59222S, S59229S, S59231S, S59232S, S59239S, S59241S, S59242S, S59249S, S59291S, S59292S, S59299S

As stated above, such an approach is time consuming and cumbersome.

Thus, the present invention is simple to use and is time saving because it starts with the relevant clinical diagnosis, uses intelligent rule-based decision making based on each placeholder, and creates relevant ICD-10 code sets based on location based, and visit-based, and severity based metadata.

Some portions of the detailed description are presented in terms of algorithms and symbolic representations of operations on data bits within a computer memory. These algorithmic descriptions and representations can be used by those skilled in the computer and software related fields.

Unless specifically stated otherwise as apparent from the following discussion, it is appreciated that throughout the description, discussions utilizing terms such as “processing” or “computing” or “calculating” or “comparing”, “generating” or “determining” or “committing” or “checkpointing” or “interrupting” or “handling” or “receiving” or “buffering” or “allocating” or “displaying” or “flagging” or Boolean logic or other set related operations or the like, refer to the action and processes of a computer system, or electronic device, that manipulates and transforms data represented as physical (electronic) quantities within the computer system's or electronic devices' registers and memories into other data similarly represented as physical quantities within electronic memories or registers or other such information storage, transmission or display devices.

The algorithms and displays presented herein are not inherently related to any particular computer or other apparatus provided it is capable of executing a rules engine. Various general purpose systems may be used with programs in accordance with the teachings herein, or it may prove convenient to construct more specialized apparatus to perform the required method steps. The required structure for a variety of these systems will appear from the description below. In addition, the present invention is not described with reference to any particular programming language, and various embodiments may thus be implemented using a variety of programming languages.

The aspects, embodiments, features, and examples of the invention are to be considered illustrative in all respects and are not intended to limit the invention, the scope of which is defined only by the claims. Other embodiments, modifications, and usages will be apparent to those skilled in the art without departing from the spirit and scope of the claimed invention.

The use of headings and sections in the application is not meant to limit the invention; each section can apply to any aspect, embodiment, or feature of the invention.

Throughout the application, where compositions are described as having, including, or comprising specific components, or where processes are described as having, including or comprising specific process steps, it is contemplated that compositions of the present teachings also consist essentially of, or consist of, the recited components, and that the processes of the present teachings also consist essentially of, or consist of, the recited process steps.

In the application, where an element or component is said to be included in and/or selected from a list of recited elements or components, it should be understood that the element or component can be any one of the recited elements or components and can be selected from a group consisting of two or more of the recited elements or components. Further, it should be understood that elements and/or features of a composition, an apparatus, or a method described herein can be combined in a variety of ways without departing from the spirit and scope of the present teachings, whether explicit or implicit herein.

The use of the terms “include,” “includes,” “including,” “have,” “has,” or “having” should be generally understood as open-ended and non-limiting unless specifically stated otherwise.

The use of the singular herein includes the plural (and vice versa) unless specifically stated otherwise. Moreover, the singular forms “a,” “an,” and “the” include plural forms unless the context clearly dictates otherwise. In addition, where the use of the term “about” is before a quantitative value, the present teachings also include the specific quantitative value itself, unless specifically stated otherwise.

It should be understood that the order of steps or order for performing certain actions is immaterial so long as the present teachings remain operable. Moreover, two or more steps or actions may be conducted simultaneously.

It is to be understood that the figures and descriptions of the invention have been simplified to illustrate elements that are relevant for a clear understanding of the invention, while eliminating, for purposes of clarity, other elements. Those of ordinary skill in the art will recognize, however, that these and other elements may be desirable. However, because such elements are well known in the art, and because they do not facilitate a better understanding of the invention, a discussion of such elements is not provided herein. It should be appreciated that the figures are presented for illustrative purposes and not as construction drawings. Omitted details and modifications or alternative embodiments are within the purview of persons of ordinary skill in the art.

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are intended to be embraced therein. cm What is claimed is:

1. A system for automating the designation of a disease classification code comprising:
a rules engine;
an input device for entering patient information in communication with the rules engine;
an output device in communication with the rules engine; and
a database in communication with the rules engine, the database comprising a plurality of rules for providing a disease classification code in response the input patient information,
wherein the rules comprise a decision tree which is traversed by the rules engine to determine the next rule and disease classification code;
wherein each of the plurality of rules comprises at least one alpha-numeric character corresponding to a digit in the disease classification code; and
wherein at least one of the plurality of rules comprises at least one alias rule comprising at least a second alpha-numeric character corresponding to a second digit in the disease classification code.

2. The system of claim 1 wherein the each rule is a render rule, a search rule or an automated rule.

3. The system of claim 2 wherein a render rule requires metadata responses.

4. The system of claim 2 wherein the search rule searches for anatomical locations.

5. The system of claim 2 wherein the disease code is generated by the selection, by a physician of a diagnosis and a location on a body may.

6. The system of claim 2 wherein the diagnosis comprises a primary diagnosis and a plurality of associated diagnoses.

7. A method for automating the designation of a disease classification code using a rules engine:
providing a rules engine;
providing a database in communication with the rules engine, the database comprising a plurality of rules for providing a disease classification code in response the input patient information
entering, by a user, patient information for use by the rules engine;
outputting a designation of disease classification from the rules engine; and
wherein the rules comprise a decision tree which is traversed by the rules engine to determine the next rule and disease classification code;
wherein each of the plurality of rules comprises at least one alpha-numeric character corresponding to a digit in the disease classification code; and
wherein at least one of the plurality of rules comprises at least one alias rule comprising at least a second alpha-numeric character corresponding to a second digit in the disease classification code.

8. The method of claim 7 wherein the each rule is a render rule, a search rule or an automated rule.

9. The method of claim 8 further comprises the step of inputting metadata responses into a render rule.

10. The method of claim 8 further comprises the step of searching for anatomical locations using a search rule.

11. The method of claim 8 further comprises generating a disease code in response to the selection, by a physician, of a diagnosis and a location on a body.

12. The method of claim 8 wherein the diagnosis comprises a primary diagnosis and a plurality of associated diagnoses.
